(19) United States
(12) Patent Application Publication
(10) Pub. No.: US 2018/0153415 A1
(43) Pub. Date: Jun. 7, 2018

(54) AORTIC STENOSIS CLASSIFICATION

(71) Applicant: Edwards Lifesciences Corporation, Irvine, CA (US)

(72) Inventors: Christine Lee, Irvine, CA (US); Feras Al Hatib, Irvine, CA (US); Camille L. Calvin, Costa Mesa, CA (US)

(73) Assignee: Edwards Lifesciences Corporation, Irvine, CA (US)

(21) Appl. No.: 15/805,446

(22) Filed: Nov. 7, 2017

Related U.S. Application Data
(60) Provisional application No. 62/429,006, filed on Dec. 1, 2016.

(51) Int. Cl. A61B 5/02; A61B 5/0215; A61B 5/00

(52) U.S. Cl. CPC A61B 5/02007; A61B 5/0215; A61B 5/742; A61B 5/6826; A61B 5/7278; A61B 5/746; A61B 5/7225

(57) ABSTRACT

According to one implementation, a medical device includes a display, a blood pressure sensor for sensing an arterial blood pressure of a patient and for generating a blood pressure signal, an analog-to-digital converter (ADC) for receiving the blood pressure signal and for converting the blood pressure signal to blood pressure data in digital form, and a hardware processor for executing an aortic stenosis diagnostic software code. The hardware processor executes the aortic stenosis diagnostic software code to receive the blood pressure data from the ADC, and to identify parameters indicative of aortic stenosis in the patient, based on the blood pressure data. The hardware processor further executes the aortic stenosis diagnostic software code to classify the severity of aortic stenosis in the patient based on an exponential function of the parameters.

### RELATED APPLICATIONS

The present application claims the benefit of and priority to a pending Provisional Patent Application Ser. No. 62/429,006, filed Dec. 1, 2016, and titled “Aortic Stenosis Classification,” which is hereby incorporated fully by reference into the present application.

### BACKGROUND

Aortic stenosis can be a progressive, debilitating, and life threatening condition if left untreated. Patients in whom aortic stenosis is present are nevertheless typically free from cardiovascular symptoms such as angina, syncope, or heart failure, for example, until late in the course of disease progression. However, once symptoms manifest, patient prognosis is often poor. As a result, early detection of aortic stenosis, prior to the manifestation of symptoms, is important.

Screening for aortic stenosis has historically been performed by cardiac auscultation, typically through use of a stethoscope to listen to a patient's heart. Although the detection of heart sounds can enable early identification of a subject suffering from aortic stenosis, there are disadvantages to relying on this conventional screening technique. One disadvantage flows from changes in the way clinicians are trained. As high technology diagnostic approaches are increasingly taught, the importance of traditional and relatively low technology diagnostic techniques may receive less emphasis, resulting in fewer diagnosticians being skilled in the use of cardiac auscultation. Another disadvantage results from the general aging of the patient population. Especially in older patients, heart sounds indicative of aortic stenosis may be present but may not reliably indicate significant aortic valvular obstruction requiring medical intervention.

### SUMMARY

There are provided systems and methods for performing aortic stenosis classification, substantially as shown in and/or described in connection with at least one of the figures, and as set forth more completely in the claims.

### BRIEF DESCRIPTION OF THE DRAWINGS

FIG. 1 shows a diagram of an exemplary aortic stenosis classification system, according to one implementation;

FIG. 2A shows an exemplary implementation for non-invasively detecting peripheral arterial blood pressure at an extremity of a patient;

FIG. 2B shows an exemplary implementation for performing minimally invasive detection of arterial blood pressure of a patient;

FIG. 3 shows a diagram depicting transformation of a peripheral arterial blood pressure waveform of a patient to a central arterial blood pressure waveform of the patient, according to one implementation;

FIG. 4 is a flowchart presenting an exemplary method for use by a system to perform aortic stenosis classification;

FIG. 5 shows a trace of an arterial blood pressure waveform including exemplary cardiac metrics;

FIG. 6 shows cross validation results of aortic stenosis classification using the methods and systems disclosed in the present application;

FIG. 7 shows the results of aortic stenosis classification using the methods and systems disclosed in the present application for subjects having mild or moderate aortic stenosis; and

FIG. 8 shows a graph of mean severity scores determined using the methods and systems disclosed in the present application for four distinct cohorts of subjects having no aortic stenosis, mild aortic stenosis, moderate aortic stenosis, and severe aortic stenosis, respectively.

### DETAILED DESCRIPTION

The following description contains specific information pertaining to implementations in the present disclosure. One skilled in the art will recognize that the present disclosure may be implemented in a manner different from that specifically discussed herein. The drawings in the present application and their accompanying detailed description are directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions.

As stated above, aortic stenosis can be a progressive, debilitating, and life threatening condition if left untreated. Patients in whom aortic stenosis is present are nevertheless typically free from cardiovascular symptoms such as angina, syncope, or heart failure, for example, until late in the course of disease progression. However, once symptoms manifest, patient prognosis is often poor. As a result, early detection of aortic stenosis, prior to the manifestation of symptoms, is important.

As also stated above, screening for aortic stenosis has historically been performed by cardiac auscultation, typically through use of a stethoscope to listen to a patient's heart. Although the detection of heart sounds can enable early identification of a subject suffering from aortic stenosis, there are disadvantages to relying on this conventional screening technique. One disadvantage flows from changes in the way clinicians are trained. As high technology diagnostic approaches are increasingly taught, the importance of traditional and relatively low technology diagnostic techniques may receive less emphasis, resulting in fewer diagnosticians being skilled in the use of cardiac auscultation. Another disadvantage results from the general aging of the patient population. Especially in older patients, heart sounds indicative of aortic stenosis may be present but may not reliably indicate significant aortic valvular obstruction requiring medical intervention.

The present application discloses systems and methods for classifying aortic stenosis in a patient that address and overcome the deficiencies associated with the conventional art noted above. The present solution for classifying aortic stenosis includes monitoring an arterial blood pressure of the patient. Such monitoring may be performed invasively, or using non-invasive arterial pressure waveform measurements taken at an extremity of the patient, for example, at a finger or wrist of the patient. In some implementations, the present solution may include applying a transfer function to transform a peripheral arterial blood pressure data detected at an extremity of the patient to a central pressure data of the patient. The present solution further includes identifying parameters that are indicative of aortic stenosis based on or using the blood pressure data, and classifying the severity of aortic stenosis based on an exponential function of those parameters.

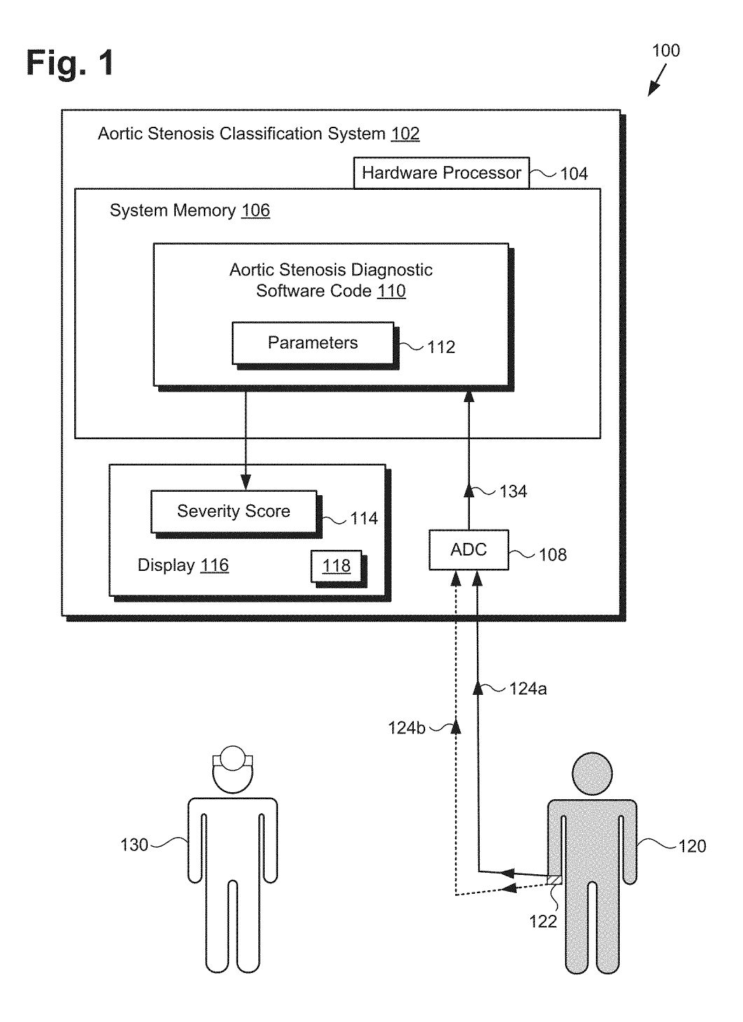

FIG. 1 shows a diagram of an exemplary aortic stenosis classification system, according to one implementation. As shown in FIG. 1, aortic stenosis classification system 102 is situated within healthcare environment 100 including patient 120, and healthcare worker 130. Aortic stenosis classification system 102 may be a medical device that includes hardware processor 104, system memory 106, analog-to-digital converter (ADC) 108 coupled to blood pressure sensor 122, display 116, and sensory alarm 118. As further shown in FIG. 1, system memory 106 stores aortic stenosis diagnostic software code 110 including parameters 112 indicative of aortic stenosis.

FIG. 1 also shows signals received by aortic stenosis classification system 102 and corresponding to an arterial blood pressure of patient 120, in the alternative as wired blood pressure signal 124a and wireless blood pressure signal 124b. In addition, FIG. 1 shows blood pressure data 134 in digital form, and aortic stenosis severity score 114 generated by aortic stenosis diagnostic software code 110 based on or using blood pressure data 134.

Blood pressure sensor 122 is shown in an exemplary implementation in FIG. 1, and is attached to patient 120. It is noted that blood pressure sensor 122 may be an invasive or non-invasive sensor attached to patient 120. In one implementation, as represented in FIG. 1, blood pressure sensor 122 may be attached non-invasively so as to sense a peripheral arterial blood pressure at an extremity of patient 120, such as arterial blood pressure measured at a wrist or finger of patient 120. Although not explicitly shown in FIG. 1, in other implementations, blood pressure sensor 122 may be attached non-invasively to measure a peripheral arterial blood pressure at another extremity of patient 120, such as at an ankle or toe of patient 120. Blood pressure signal 124a/124b received by ADC 108 of aortic stenosis classification system 102 may include a central or peripheral arterial blood pressure waveform of patient 120.

According to one exemplary implementation, aortic stenosis classification system 102 may correspond to one or more web servers, accessible over a packet-switched network such as the Internet, for example. In another implementation, aortic stenosis classification system 102 may correspond to one or more servers supporting a local area network (LAN), or included in another type of limited distribution network, such as within a hospital setting, for example. In yet other implementations, aortic stenosis classification system 102 may take the form of a computer workstation or personal computer (PC), a dedicated handheld or otherwise portable diagnostic system, or any type of mobile computing device, such as a smartphone or tablet computer, among others.

According to the exemplary implementation shown in FIG. 1, hardware processor 104 is configured to utilize ADC 108 to convert blood pressure signal 124a/124b to blood pressure data 134 in digital form. Hardware processor 104 is also configured to execute aortic stenosis diagnostic software code 110 to receive blood pressure data 134 from ADC 108. In addition, in some implementations, hardware processor 104 may be further configured to execute aortic stenosis diagnostic software code 110 to apply a transfer function for transforming blood pressure data 134 to central pressure data corresponding to a central arterial blood pressure of patient 120. For example, where blood pressure signal 124a/124b is provided by non-invasive finger or wrist arterial pressure sensor 122, aortic stenosis diagnostic software code 110 may be used to apply a transfer function for transforming blood pressure data 134 corresponding to a peripheral arterial pressure of patient 120 to an aortic blood pressure or a brachial blood pressure of patient 120.

Hardware processor 104 is also configured to execute aortic stenosis diagnostic software code 110 to extract or otherwise identify parameters 112 indicative of aortic stenosis in patient 120 based on blood pressure data 134 or using blood pressure data 134 when blood pressure data 134 includes the central pressure data of patient 120. In addition, hardware processor 104 is configured to execute aortic stenosis diagnostic software code 110 to determine severity score 114 for classifying aortic stenosis in patient 120 based on parameters 112.

It is noted that severity score 114, when generated, may be stored in system memory 106, may be copied to non-volatile storage (not shown in FIG. 1), or may be displayed to healthcare worker 130 on display 116 of aortic stenosis classification system 102. Display 116 may take the form of a liquid crystal display (LCD), a light-emitting diode (LED) display, an organic light-emitting diode (OLED) display, or another suitable display screen that performs a physical transformation of signals to light.

It is further noted that hardware processor 104 may execute aortic stenosis diagnostic software code 110 to activate sensory alarm 118 if severity score 114 meets or exceeds a predetermined threshold value, that is to say, based on the severity of aortic stenosis in patient 120. In various implementations, sensory alarm 118 may include one or more of a visual alarm, an audible alarm, and a haptic alarm. For example, when implemented to provide a visual alarm, sensory alarm 118 may be activated as flashing and/or colored graphics shown on display 116. When implemented to provide an audible alarm, sensory alarm 118 may be activated as any suitable warning sound, such as a siren or repeated tone. Moreover, when implemented to provide a haptic alarm, sensory alarm 118 may cause one or more components of aortic stenosis classification system 102 to vibrate or otherwise deliver a physical impulse perceptible to healthcare worker 130.

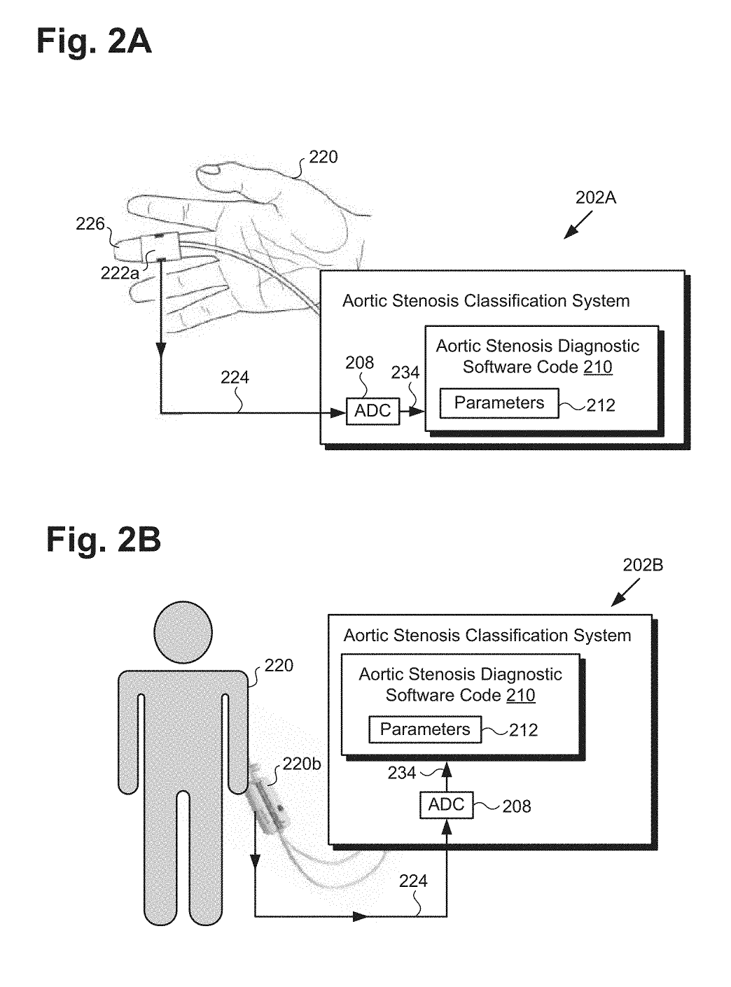

FIG. 2A shows an exemplary implementation for sensing peripheral arterial blood pressure non-invasively at an extremity of a patient. Aortic stenosis classification system 202A, in FIG. 2A, includes ADC 208 and aortic stenosis diagnostic software code 210. As shown by FIG. 2A, the arterial blood pressure of patient 220 is sensed non-invasively at finger 226 of patient 220 using blood pressure sensing cuff 222a. Also shown in FIG. 2A are blood pressure signal 224 received by ADC 208 of aortic stenosis classification system 202A from blood pressure sensing cuff 222a, digital blood pressure data 234 converted from blood pressure signal 224 by ADC 208, and parameters 212 indicative of aortic stenosis in patient 220, and identified based on blood pressure data 234 by aortic stenosis diagnostic software code 210.

Patient 220, blood pressure signal 224, and digital blood pressure data 234 correspond respectively in general to patient 120, blood pressure signal 124a/124b, and digital blood pressure data 134, in FIG. 1, and those corresponding features may share the characteristics attributed to any corresponding feature by the present disclosure. Moreover, aortic stenosis classification system 202A including blood pressure sensing cuff 222a, ADC 208, and aortic stenosis diagnostic software code 210 including parameters 212, in FIG. 2A, corresponds in general to aortic stenosis classification system 102 including blood pressure sensor 122, ADC 108, and aortic stenosis diagnostic software code 110 including parameters 112, in FIG. 1, and those corresponding features may share any of the characteristics attributed to either corresponding feature by the present disclosure. In other words, although not explicitly shown in FIG. 2A, aortic stenosis classification system 202A includes features corresponding respectively to hardware processor 104, display 116, and sensory alarm 118.

According to the implementation shown in FIG. 2A, blood pressure sensing cuff 222a is designed to sense a peripheral arterial blood pressure of patient 120/220 non-invasively at finger 226 of patient 120/220. Moreover, as shown in FIG. 2A, blood pressure sensing cuff 222a may take the form of a small, lightweight, and comfortable blood pressure sensor suitable for extended wear by patient 120/220. It is noted that although blood pressure sensing cuff 222a is shown as a finger cuff, in FIG. 2A, in other implementations, blood pressure sensing cuff 222a may be suitably adapted as a wrist, ankle, or toe cuff for attachment to patient 120/220.

It is further noted that the advantageous extended wear capability described above for blood pressure sensing cuff 222a when implemented as a finger cuff may also be attributed to wrist, ankle, and toe cuff implementations. As a result, blood pressure sensing cuff 222a may be configured to provide substantially continuous beat-to-beat monitoring of the peripheral arterial blood pressure of patient 120/220 over an extended period of time, such as minutes or hours, for example.

FIG. 2B shows an exemplary implementation for performing minimally invasive detection of arterial blood pressure of a patient. As shown by FIG. 2B, the radial arterial blood pressure of patient 120/220 is detected via minimally invasive blood pressure sensor 222b. It is noted that the features shown in FIG. 2B and identified by reference numbers identical to those shown in FIG. 2A correspond respectively to those previously described features, and may share any of the characteristics attributed to them above. It is further noted that blood pressure sensor 222b corresponds in general to blood pressure sensor 122, in FIG. 1, and those corresponding features may share any of the characteristics attributed to either corresponding feature by the present disclosure.

According to the implementation shown in FIG. 2B, blood pressure sensor 222b is designed to sense an arterial blood pressure of patient 120/220 in a minimally invasive manner. For example, blood pressure sensor 222b may be attached to patient 120/220 via a radial arterial catheter inserted into an arm of patient 120/220. Alternatively, and although not explicitly represented in FIG. 2B, in another implementation, blood pressure sensor 222b may be attached to patient 120/220 via a femoral arterial catheter inserted into a leg of patient 120/220. Like non-invasive blood pressure sensing cuff 222a, in FIG. 2A, minimally invasive blood pressure sensor 222b, in FIG. 2B, may be configured to provide substantially continuous beat-to-beat monitoring of the arterial blood pressure of patient 120/220 over an extended period of time, such as minutes or hours.

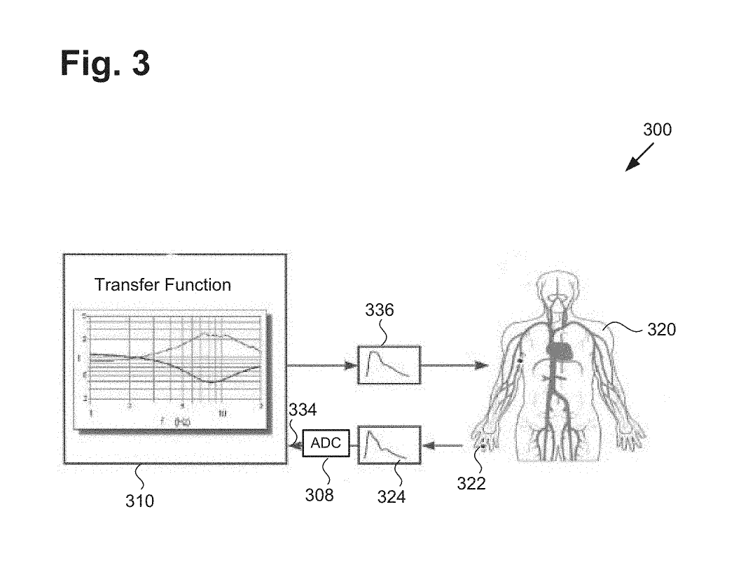

FIG. 3 shows diagram 300 depicting transformation of digital blood pressure data 334, converted by ADC 308 from blood pressure signal 324, to central pressure data 336, according to one implementation. Also shown in FIG. 3 are patient 320, blood pressure sensor 322, and aortic stenosis diagnostic software code 310. Blood pressure sensor 322, blood pressure signal 324, ADC 308, digital blood pressure data 334, and aortic stenosis diagnostic software code 310 correspond respectively in general to blood pressure sensor 122/222a/222b, blood pressure signal 124a/124b/224, ADC 108/208, digital blood pressure data 134/234, and aortic stenosis diagnostic software code 110/210, in FIGS. 1, 2A, and 2B, and those corresponding features may share the characteristics attributed to any corresponding feature by the present disclosure.

Thus, blood pressure signal 324 and digital blood pressure data 334 can correspond to a peripheral arterial blood pressure of patient 120/220/320 detected using blood pressure sensor 122/222a/222b/322. As shown in FIG. 3, digital blood pressure data 134/234/334, converted from blood pressure signal 124a/124b/224/324 by ADC 108/208/308, may be transformed to central pressure data 336 of patient 120/220/320. As further shown by FIG. 3, such a transformation may be performed by aortic stenosis diagnostic software code 110/210/310 through application of a transfer function to digital blood pressure data 134/234/334. That is to say, application of such a transfer function may be performed by aortic stenosis diagnostic software code 110/210/310, executed by hardware processor 104.

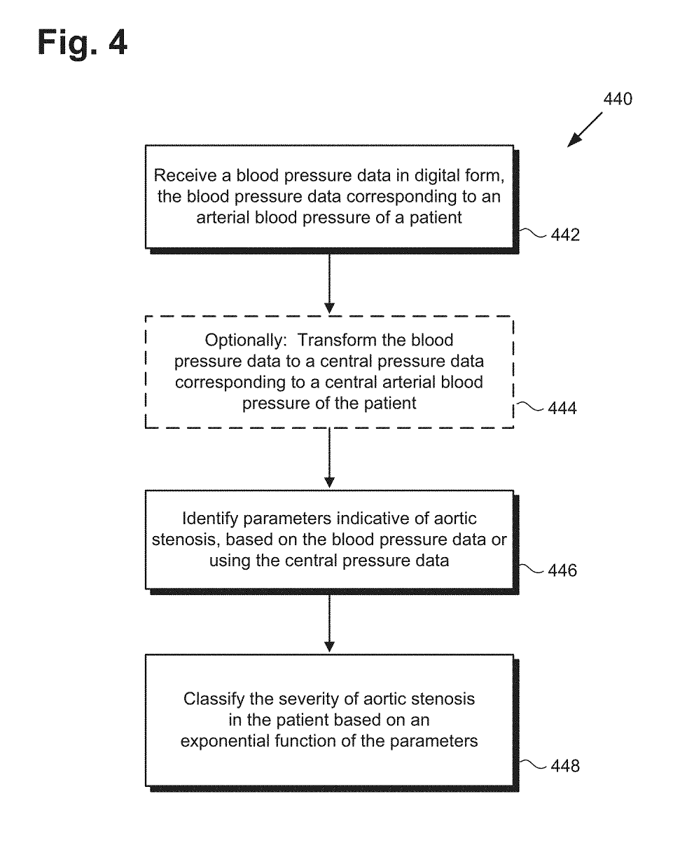

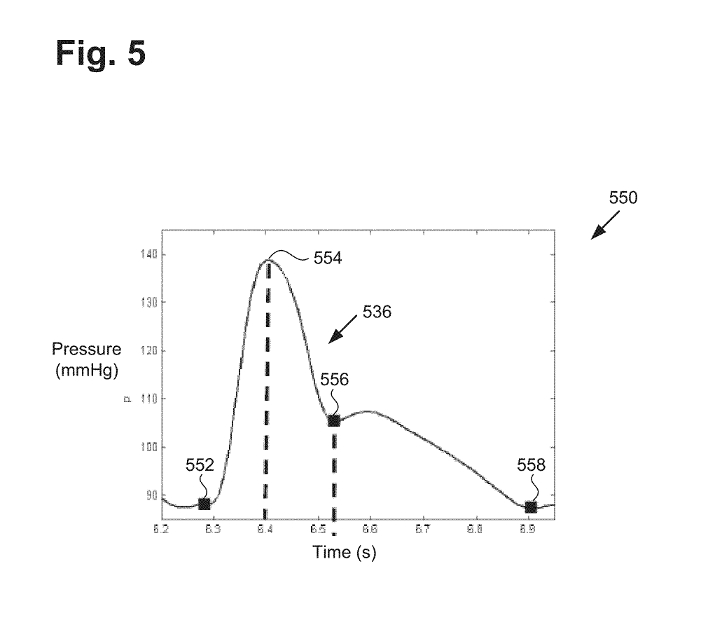

Example implementations of the present inventive principles will be further described below with reference to FIGS. 4 and 5. FIG. 4 presents flowchart 440 outlining an exemplary method for use by a system to perform aortic stenosis classification. FIG. 5 shows a trace of a central arterial blood pressure waveform including exemplary cardiac metrics.

Referring to FIG. 4 in combination with FIGS. 1, 2A, 2B, and 3, flowchart 440 begins with receiving blood pressure data 134/234/334 in digital form (action 442). As noted above, blood pressure sensor 122/222a/222b/322 may sense an arterial blood pressure of patient 120/220/320 and may generate blood pressure signal 124a/124b/224/324. As further noted above, ADC 108/208/308 of aortic stenosis classification system 102/202A/202B may receive blood pressure signal 124a/124b/224/324 from blood pressure sensor 122/222a/222b/322, and may convert blood pressure signal 124a/124b/224/324 to blood pressure data 134/234/334 in digital form. Blood pressure data 134/234/334 may be received by aortic stenosis diagnostic software code 110/210/310, executed by hardware processor 104.

In some implementations, blood pressure sensor 122/222a/222b/322 may be used to sense a central arterial blood pressure of patient 120/220/320, and to generate blood pressure signal 124a/124b/224/324 as an analog signal corresponding to that central arterial blood pressure. In those implementations, blood pressure data 134/234/334 may be substantially identical to central pressure data 336 of patient 120/220/320, and may be used to identify parameters 112/212 indicative of aortic stenosis. However, in other implementations, blood pressure sensor 122/222a/222b/322 may be used to sense a peripheral arterial blood pressure of patient 120/220/320, and to generate blood pressure signal 124a/124b/224/324 as an analog signal corresponding to that peripheral arterial blood pressure.

In implementations in which blood pressure sensor 122/222a/222b/322 is used to sense a peripheral arterial blood pressure of patient 120/220/320, flowchart 440 may include transforming blood pressure data 134/234/334 to central pressure data 336 of patient 120/220/320 (action 444). Central pressure data 336 may include a central blood pressure waveform of patient 120/220/320, such as an aortic blood pressure waveform of patient 120/220/320, for example. The optional transformation of blood pressure data 134/234/334 to central pressure data 336 may be performed by aortic stenosis diagnostic software code 110/210/310, executed by hardware processor 104, in the manner described above by reference to FIG. 3.

Flowchart 440 continues with extracting or otherwise identifying parameters 112/212 indicative of aortic stenosis based on blood pressure data 134/234/334, or using blood pressure data 134/234/334 (action 446). As noted above, in implementations in which blood pressure data 134/234/334 is converted from blood pressure signal 124a/124b/224/324 corresponding to a peripheral arterial blood pressure of patient 120/220/320, blood pressure data 134/234/334 may be converted to central pressure data 336 for use in identifying parameters 112/212. Thus, in those implementations, parameters 112/212 are identified based on blood pressure data 134/234/334 and using central pressure data 336.

However, as also noted above, in implementations in which blood pressure data 134/234/334 is converted from blood pressure signal 124a/124b/224/324 corresponding to a central arterial blood pressure of patient 120/220/320, blood pressure data 134/234/334 may be substantially identical to central pressure data 336 of patient 120/220/320 without transformation. Thus, in those implementations, parameters 112/212 may be identified using blood pressure data 134/234/334 directly. Whether identified based on blood pressure data 134/234/334, or using blood pressure data 134/234/334 directly, parameters 112/212 may be identified by aortic stenosis diagnostic software code 110/210/310, executed by hardware processor 104.

Referring to FIG. 5, FIG. 5 shows trace 550 of exemplary central arterial blood pressure waveform 536 corresponding to central pressure data 336, in FIG. 3. As shown in FIG. 5, central arterial blood pressure waveform 536 is expressed as a function of time, and includes heartbeat metrics 552, 554, 556, and 558. Heartbeat metrics 552, 554, 556, and 558 correspond respectively to the start of a heartbeat, the maximum systolic pressure marking the end of systolic rise, the presence of the dicrotic notch marking the end of systolic decay, and the beginning of the next heartbeat of patient 120/220/320. Heartbeat metrics 552, 554, 556, and 558 may be included among parameters 112/212 indicative of aortic stenosis and identified by aortic stenosis diagnostic software code 110/210/310.

It is noted that although heartbeat metrics 552, 554, 556, and 558 are shown for conceptual clarity, more generally, parameters 112/212 indicative of aortic stenosis in patient 120/220/320 may include a variety of different types of parameters, some of which may include and/or be based on heartbeat metrics 552, 554, 556, and 558. For instance, parameters 112/212 indicative of aortic stenosis may include any or all of mean arterial pressure (MAP), combinatorial parameters, hemodynamic complexity parameters, and frequency domain hemodynamic parameters.

Hemodynamic complexity parameters quantify the amount of regularity in cardiac measurements over time, as well as the entropy, i.e., the unpredictability of fluctuations in cardiac measurements over time. Frequency domain hemodynamic parameters quantify various measures of cardiac performance as a function of frequency rather than time.

In some implementations, blood pressure signal 124a/124b/224/244 corresponding to an arterial blood pressure of patient 120/220/320 may be periodically, or substantially continuously monitored by aortic stenosis classification system 102/202A/202B during a sampling interval lasting several minutes, such as fifteen minutes, for example. Moreover, during that sampling interval, the parameters 112/212 indicative of aortic stenosis may be averaged repeatedly using sampling periods of several seconds, such as twenty seconds, for example. In other words, in an exemplary implementation in which parameters 112/212 indicative of aortic stenosis are sampled and averaged repeatedly for twenty seconds over a fifteen minute sampling interval, forty five distinct data points can be collected for each of parameters 112/212.

Flowchart 440 can conclude with classifying the severity of aortic stenosis in patient 120/220/320 based on an exponential function of parameters 112/212 (action 448). Classification of the severity of aortic stenosis in patient 120/220/320 may be performed by aortic stenosis diagnostic software code 110/210/310, executed by hardware processor 104, and may be expressed as severity score 114.

In classifying the severity of aortic stenosis in patient 120/220/320, it may be advantageous or desirable to place greater emphasis on some parameters 112/212 indicative of aortic stenosis than on others when determining severity score 114. In other words, in some implementations, aortic stenosis diagnostic software code 110/210/310, executed by hardware processor 104, may use a weighted combination of parameters 112/212 to determine severity score 114. Moreover, it is noted that the weighting factors applied respectively to parameters 112/212 may by positive or negative.

In one implementation, for example, the exponential function on which determination of severity score 114, and thus classification of aortic stenosis in patient 120/220/320, is based may be an exponential function of a weighted sum of parameters 112/212. Moreover, in implementations in which parameters 112/212 are monitored during a sampling interval lasting several minutes, as described above, classification of the severity of aortic stenosis in patient 120/220/320 may include identifying an average value for each of parameters 112/212 during the sampling interval. In those implementations, the exponential function on which determination of severity score 114 is based may be an exponential function of a weighted sum of the average values of parameters 112/212.

It is emphasized that severity score 114 for patient 120/220/320 is determined based on a weighted combination of parameters 112/212 identified based on or using blood pressure data 134/234/334 corresponding to an arterial blood pressure of patient 120/220/320. Consequently, according to the inventive concepts disclosed by the present application, hardware processor 104 of aortic stenosis classification system 102/202A/202B is configured to execute aortic stenosis diagnostic software code 110/210/310 to determine severity score 114 for patient 120/220/320 without direct comparison with data corresponding to aortic stenosis in other patients or research subjects.

Thus, aortic stenosis diagnostic software code 110/210/310 determines severity score 114 for subject 120/220/320 based on parameters 112/212 identified based on or using blood pressure data 134/234/334, without reference to a database storing information regarding aortic stenosis in patients or research subjects other than patient 120/220/320. Moreover, execution of aortic stenosis diagnostic software code 110/210/310 by hardware processor 104 can substantially automate determination of severity score 114, and hence aortic stenosis classification.

By way merely of example, according to one implementation, severity score 114 may be expressed as:

Severity Score=1/(1+e^{-(bias+Σβx)}) (Equation 1)

Where:

Σβx=w₁×x₁+w₂×x₂+w₃×x₃+w₄×x₄+w₅×x₅+w₆×x₆+w₇×x₇+w₈×x₈+w₉×x₉+w₁₀×x₁₀+w₁₁×x₁₁+w₁₂×x₁₂

In other words, in the present example, Σβx is the weighted sum of twelve parameters 112/212, i.e., “xᵢ” (i=1, 2, 3, . . . , 12), identified as indicative of aortic stenosis, where the contribution of each parameter to the summation is determined by its respective weighting factor “wⱼ” (j=1, 2, 3, . . . , 12).

According to one example implementation:
bias=0.99
w₁=1.21
w₂=0.13
w₃=0.06
w₄=0.05
w₅=0.03
w₆=−0.01
w₇=−0.07
w₈=−0.19
w₉=−0.28
w₁₀=−0.54
w₁₁=−0.58
w₁₂=−1.17

And parameters 112/212 include:
x₁=Cardiac output
x₂=Entropy of mean arterial blood pressure (MAP)
x₃=Entropy of the systolic pressure minus dicrotic notch pressure
x₄=Entropy of duration of the diastolic phase: the time from the dicrotic notch to the start of the next beat
x₅=The skewness of the pressure waveform within a beat
x₆=Entropy of stroke volume
x₇=Time from the first beat sample exceeding the beat mean to the dicrotic notch
x₈=Vascular tone computed with a balanced multivariate model derived from patients with mild hyperdynamic conditions
x₉=Cardiac work index
x₁₀=Cardiac index
x₁₁=Ratio of heart rate to the systolic blood pressure
x₁₂=Arterial tone estimate

In some implementations, severity score 114 may be expressed as a fraction, as represented by Equation 1. However, in other implementations, severity score 114 may be converted to a percentage score between zero percent and one hundred percent. In addition, in some implementations, as shown by FIG. 1, hardware processor 104 may further execute aortic stenosis diagnostic software code 110/210/310 to output severity score 114 to display 116 of aortic stenosis classification system 102/202A/202B.

As noted above, in some implementations, hardware processor 104 may further execute aortic stenosis diagnostic software code 110 to activate sensory alarm 118 based on the severity of aortic stenosis in patient 120/220/320. For example, hardware processor 104 may further execute aortic stenosis diagnostic software code 110 to activate sensory alarm 118 if severity score 114 meets or exceeds a predetermined threshold value.

As also noted above, in various implementations, sensory alarm 118 may include one or more of a visual alarm, an audible alarm, and a haptic alarm. For example, when implemented to provide a visual alarm, sensory alarm 118 may be activated as flashing and/or colored graphics shown on display 116. When implemented to provide an audible alarm, sensory alarm 118 may be activated as any suitable warning sound, such as a siren or repeated tone. Moreover, when implemented to provide a haptic alarm, sensory alarm 118 may cause one or more components of aortic stenosis classification system 102 to vibrate or otherwise deliver a physical impulse perceptible to healthcare worker 130.

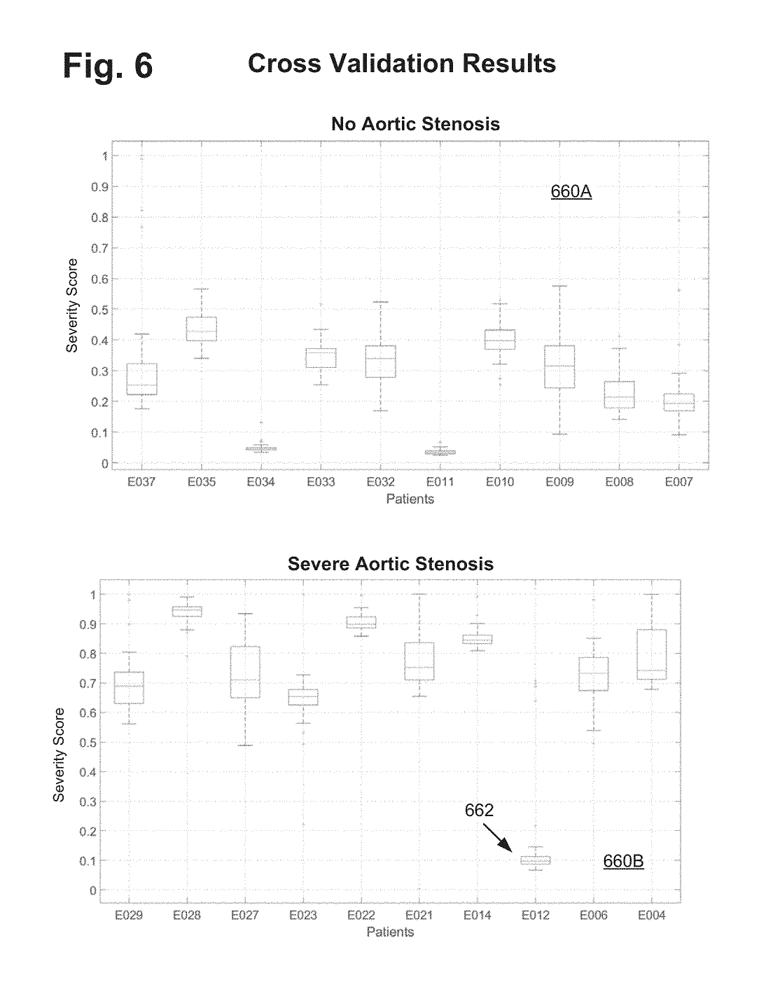

FIG. 6 shows cross validation results of aortic stenosis classification using the methods and systems disclosed in the present application. Graph 660A presents the distribution of severity scores 114 for a cohort of subjects for whom aortic stenosis is not present. In addition, graph 660B presents an analogous distribution of severity scores 114 for another cohort of subjects diagnosed with severe aortic stenosis. The severity score distributions shown in FIG. 6 were determined across fifteen minutes of data collection for each subject, in other words, during a fifteen minute sampling interval for each subject. It is noted that the reference subjects for the research resulting in the graphs shown in FIGS. 6, 7, and 8 are referred to as “subjects” rather than patients because at least some of those subjects may be voluntary research participants, rather than patients undergoing diagnosis and/or receiving treatment.

As shown in FIG. 6, all subjects for whom aortic stenosis is not present were determined to have severity scores of less than 0.5. By contrast, most subjects having severe aortic stenosis were determined to have severity scores above 0.6, with many subjects having severity scores substantially higher than 0.6. Thus, based on severity score 114, aortic stenosis may be classified as mild, moderate or severe. For example, severity score 114 of less than 0.3 may indicate a mild aortic stenosis, severity score 114 of between 0.3 and 0.6 may indicate a moderate aortic stenosis, and severity score 114 of more than 0.6 may indicate a severe aortic stenosis.

In moderate cases of aortic stenosis, echocardiography may be performed on the patient every 1-2 years to monitor the progression, possibly complemented with a cardiac stress test. In severe cases of aortic stenosis, echocardiography may be performed on the patient every 3-6 months. Also, in adult patients, a symptomatic severe aortic stenosis usually requires aortic valve replacement (AVR). While AVR has been the standard of care for aortic stenosis for several decades, other options to AVR include open heart surgery, minimally invasive cardiac surgery (MICS) and minimally invasive catheter-based aortic valve replacement. For infants and children, balloon valvuloplasty may be used, where a balloon is inflated to stretch the valve and allow greater flow. Thus, in response to classification of severity score 114, the patient having an increased risk for death may be treated within a sufficient lead time to decrease the patient's risk of death.

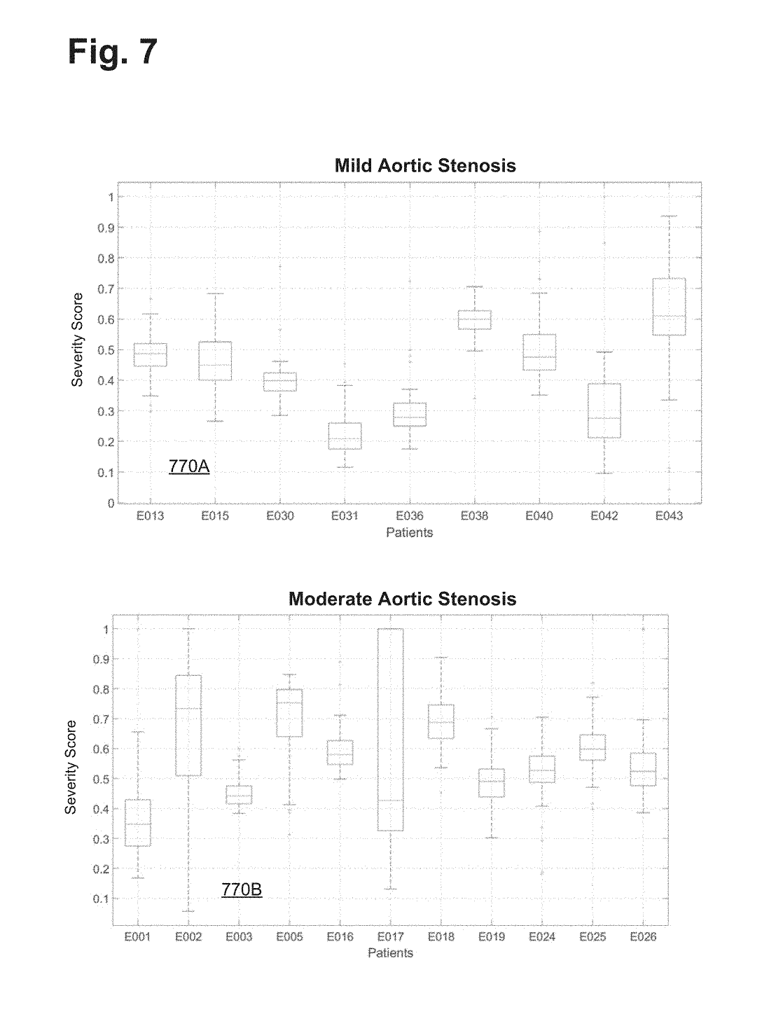

FIG. 7 shows the results of aortic stenosis classification using the methods and systems disclosed in the present application for subjects having mild or moderate aortic stenosis. Graph 770A presents the distribution of severity scores 114 for subjects having mild aortic stenosis, while graph 770B presents an analogous distribution of severity scores 114 for subjects having moderate aortic stenosis.

It is noted that the severity score distributions shown in FIG. 7 were determined across fifteen minutes of data collection for each subject in other words, during a fifteen minute sampling interval for each subject. It is further noted that none of the subjects represented in graph 770a or 770b was used to generate the cross validation results shown in FIG. 6, or to train the classification model used by aortic stenosis diagnostic software code 110/210/310. As shown in FIG. 7, there is a statistically significant separation between subjects having moderate aortic stenosis and those having mild aortic stenosis, with the severity scores of those with moderate aortic stenosis trending higher than those of subjects having mild aortic stenosis.

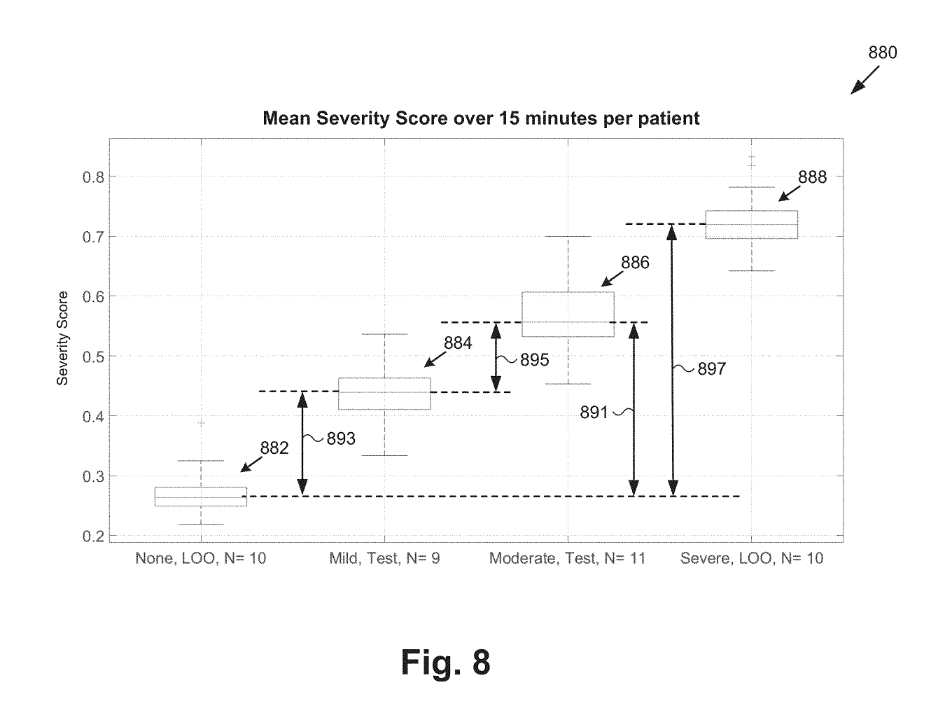

FIG. 8 shows graph 880 of mean severity scores 114 determined using the methods and systems disclosed in the present application for four distinct cohorts of subjects 882, 884, 886, and 888 having no aortic stenosis, mild aortic stenosis, moderate aortic stenosis, and severe aortic stenosis, respectively. The mean severity score distributions shown in FIG. 8 were determined across fifteen minutes of data collection for each subject in other words, during a fifteen minute sampling interval for each subject.

As shown in FIG. 8, there is a statistically significant difference 893 between the mean severity score for cohort of subjects 882 having no aortic stenosis and the mean severity score for cohort of subjects 884 having mild aortic stenosis. As further shown in FIG. 8, there are also statistically significant differences 895 and 891 between the mean severity score for cohort of subjects 886 having moderate aortic stenosis and the respective mean severity scores for cohort of subjects 884 having mild aortic stenosis and cohort of subjects 882 having no aortic stenosis. Moreover, graph 880 shows additional statistically significant difference 897 between the mean severity score for cohort of subjects 888 having severe aortic stenosis and the mean severity score for cohort of subjects 882 having no aortic stenosis.

Thus, by substantially automating aortic stenosis classification, the solution disclosed by the present application advantageously enables early detection of aortic stenosis by clinicians having little or no expertise in cardiac auscultation. In addition, by enabling performance of aortic stenosis diagnosis and classification based on arterial blood pressure measurements obtained non-invasively or minimally invasively from a to patient, the methods and systems disclosed in the present application advantageously enhance patient comfort and safety. Moreover, by enabling substantially continuous beat-to-beat monitoring of arterial blood pressure at an extremity of the patient, such as at the patient's finger, the present application discloses a compact, portable aortic stenosis classification solution suitable for deployment to cardiology offices or primary care sites.

From the above description it is manifest that various techniques can be used for implementing the concepts described in the present application without departing from the scope of those concepts. Moreover, while the concepts have been described with specific reference to certain implementations, a person of ordinary skill in the art would recognize that changes can be made in form and detail without departing from the scope of those concepts. As such, the described implementations are to be considered in all respects as illustrative and not restrictive. It should also be understood that the present application is not limited to the particular implementations described herein, but many rearrangements, modifications, and substitutions are possible without departing from the scope of the present disclosure.

What is claimed is:

1. A medical device comprising:
a display;
a blood pressure sensor configured to sense an arterial blood pressure of a patient and generate a blood pressure signal;
an analog-to-digital converter (ADC) configured to receive the blood pressure signal and convert the blood pressure signal to blood pressure data in digital form;
a hardware processor configured to execute an aortic stenosis diagnostic software code to:
receive the blood pressure data from the ADC;
identify a plurality of parameters indicative of aortic stenosis in the patient, based on the blood pressure data; and
classify a severity of aortic stenosis in the patient based on an exponential function of the plurality of parameters.

2. The medical device of claim 1, wherein in response to classifying the severity of aortic stenosis in the patient, the patient having an increased risk for death is treated within a sufficient lead time to decrease the patient's risk of death.

3. The medical device of claim 1, wherein the hardware processor is further configured to execute the aortic stenosis diagnostic software code to output a severity score corresponding to the severity of aortic stenosis in the patient to the display.

4. The medical device of claim 1, wherein the hardware processor is further configured to execute the aortic stenosis diagnostic software code to activate a sensory alarm based on the severity of aortic stenosis in the patient.

5. The medical device of claim 1, wherein the hardware processor is further configured to execute the aortic stenosis diagnostic software code to:
before classifying the severity of aortic stenosis in the patient, monitor the plurality of parameters indicative of aortic stenosis in the patient during a sampling interval lasting a plurality of minutes; and
identify an average value for each of the plurality of parameters during the sampling interval;
wherein the exponential function of the plurality of parameters is an exponential function of the average values.

6. The medical device of claim 1, wherein the exponential function of the plurality of parameters is an exponential function of a weighted sum of the average values.

7. The medical device of claim 1, wherein the blood pressure sensor is configured to sense the arterial blood pressure of the patient invasively.

8. The medical device of claim 1, wherein the blood pressure sensor is configured to sense the arterial blood pressure of the patient non-invasively.

9. The medical device of claim 1, wherein the blood pressure sensor is configured to sense a peripheral arterial blood pressure of the patient non-invasively at an extremity of the patient.

10. The medical device of claim 9, wherein the hardware processor is further configured to execute the aortic stenosis diagnostic software code to:
before identifying the plurality of parameters indicative of aortic stenosis in the patient, transform the blood pressure data to a central pressure data corresponding to a central arterial blood pressure of the patient, and
identify the plurality of parameters using the central pressure data.

11. A method of using a medical device including a display a blood pressure sensor, an analog-to-digital converter (ADC), and a hardware processor, the method comprising:
sensing, using the blood pressure sensor, an arterial blood pressure of a patient and generating a blood pressure signal;
converting, using the ADC, the blood pressure signal to blood pressure data in digital form;
receiving, using the hardware processor, the blood pressure data from the ADC;
identifying, using the hardware processor, a plurality of parameters indicative of aortic stenosis in the patient, based on the blood pressure data; and
classifying, using the hardware processor, a severity of aortic stenosis in the patient based on an exponential function of the plurality of parameters.

12. The method of claim 11 further comprising:
in response to the classifying of the severity of aortic stenosis in the patient, treating the patient having an increased risk for death within a sufficient lead time to decrease the patient's risk of death.

13. The method of claim 11, further comprising outputting, using the hardware processor, a severity score corresponding to the severity of aortic stenosis in the patient to the display.

14. The method of claim 11, further comprising activating, using the hardware processor, a sensory alarm based on the severity of aortic stenosis in the patient.

15. The method of claim 11, further comprising:
before classifying the severity of aortic stenosis in the patient, monitoring, using the hardware processor, the plurality of parameters indicative of aortic stenosis in the patient during a sampling interval lasting a plurality of minutes; and
identifying, using the hardware processor, an average value for each of the plurality of parameters during the sampling interval;
wherein the exponential function of the plurality of parameters is an exponential function of the average values.

16. The method of claim 11 wherein the exponential function of the plurality of parameters is an exponential function of a weighted sum of the average values.

17. The method of claim 11, further comprising sensing the arterial blood pressure of the patient invasively using the blood pressure sensor.

18. The method of claim 11, further comprising sensing the arterial blood pressure of the patient non-invasively using the blood pressure sensor.

19. The method of claim 11, further comprising sensing a peripheral arterial blood pressure of the patient non-invasively at an extremity of the patient, using the blood pressure sensor.

20. The method of claim 19, further comprising:
before identifying the plurality of parameters indicative of aortic stenosis in the patient, transforming, using the hardware processor, the blood pressure data to a central pressure data corresponding to a central arterial blood pressure of the patient, and
identifying, using the hardware processor, the plurality of parameters using the central pressure data.
